SEQUENCE LISTING (28 sequences)

SEQ ID NO: 1 - protein, 238 aa, Artificial Sequence
  Description of Artificial Sequence antibody or antibody fragment
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 2 - protein, 4 aa, Artificial Sequence
  Description of Artificial Sequenceantibody or antibody fragment
XXXC

SEQ ID NO: 3 - protein, 5 aa, Artificial Sequence
  Description of Artificial Sequence antibody or antibody fragment
XXXCX

SEQ ID NO: 4 - protein, 6 aa, Artificial Sequence
  Description of Artificial Sequence antibody or antibody fragment
HHHHHH

SEQ ID NO: 5 - protein, 17 aa, Artificial Sequence
  Description of Artificial Sequence antibody or antibody fragment
AAADDDSDDDYKDDDDK

SEQ ID NO: 6 - protein, 23 aa, Artificial Sequence
  Description of Artificial Sequence antibody or antibody fragment
AAADDDSDDDYKDDDDKHHHHHH

SEQ ID NO: 7 - protein, 121 aa, Artificial Sequence
  Description of Artificial Sequence antibody or antibody fragment
SGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 8 - protein, 5 aa, Artificial Sequence
  Description of Artificial Sequence antibody or antibody fragment
SFSMS

SEQ ID NO: 9 - protein, 17 aa, Artificial Sequence
  Description of Artificial Sequence antibody or antibody fragment
SISGSSGTTYYADSVKG

SEQ ID NO: 10 - protein, 7 aa, Artificial Sequence
  Description of Artificial Sequence antibody or antibody fragment
PFPYFDY

SEQ ID NO: 11 - protein, 12 aa, Artificial Sequence
  Description of Artificial Sequence antibody or antibody fragment
RASQSVSSSFLA

SEQ ID NO: 12 - protein, 6 aa, Artificial Sequence
  Description of Artificial Sequence antibody or antibody fragment
YASSRA

SEQ ID NO: 13 - protein, 10 aa, Artificial Sequence
  Description of Artificial Sequence antibody or antibody fragment
CQQTGRIPPT

SEQ ID NO: 14 - protein, 4 aa, Artificial Sequence
  Description of Artificial Sequence antibody or antibody fragment
GGGC

SEQ ID NO: 15 - protein, 4 aa, Artificial Sequence
  Description of Artificial Sequence antibody or antibody fragment
GCGC

SEQ ID NO: 16 - protein, 5 aa, Artificial Sequence
  Description of Artificial Sequence antibody or antibody fragment
GGGCA

SEQ ID NO: 17 - protein, 5 aa, Artificial Sequence
  Description of Artificial Sequence antibody or antibody fragment
GCGCA

SEQ ID NO: 18 - protein, 6 aa, Artificial Sequence
  Description of Artificial Sequence antibody or antibody fragment
HHHHHH

SEQ ID NO: 19 - protein, 22 aa, Artificial Sequence
  Description of Artificial Sequence antibody or antibody fragment
MKYLLPTAAAGLLLLAAQPAMA

SEQ ID NO: 20 - protein, 242 aa, Artificial Sequence
  Description of Artificial Sequence antibody or antibody fragment
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGC

SEQ ID NO: 21 - protein, 243 aa, Artificial Sequence
  Description of Artificial Sequence antibody or antibody fragment
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGCA

SEQ ID NO: 22 - protein, 242 aa, Artificial Sequence
  Description of Artificial Sequence antibody or antibody fragment
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGCGC

SEQ ID NO: 23 - protein, 243 aa, Artificial Sequence
  Description of Artificial Sequence antibody or antibody fragment
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGCGCA

SEQ ID NO: 24 - protein, 283 aa, Artificial Sequence
  Description of Artificial Sequence antibody or antibody fragment
MKYLLPTAAAGLLLLAAQPAMAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAAADDDSDDDYKDDDDKHHHHHH

SEQ ID NO: 25 - protein, 365 aa, Artificial Sequence
  Description of Artificial Sequence antibody or antibody fragment
MXDTPAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 26 - protein, 244 aa, Artificial Sequence
  Description of Artificial Sequence antibody or antibody fragment
MXDTPAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 27 - protein, 243 aa, Artificial Sequence; identical to SEQ ID NO: 21
  Description of Artificial Sequence antibody or antibody fragment

SEQ ID NO: 28 - protein, 5 aa, Artificial Sequence
  Description of Artificial Sequence antibody or antibody fragment
HHHHH

(19) United States
(12) Patent Application Publication
(10) Pub. No.: US 2005/0112690 A1
(43) Pub. Date: May 26, 2005

(54) Binding molecules for the extra-domain B of fibronectin for detection of arteriosclerotic plaque

(76) Inventor: Dieter Heldmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/966,097

(22) Filed: Oct. 18, 2004

(30) Foreign Application Priority Data
Oct. 17, 2003 (DE) 10348319.5

(51) Int. Cl. G01N 33/53

(52) U.S. Cl. 435/7.1

(57) ABSTRACT

This invention relates to the use of labeled L19 derivatives for the production of a pharmaceutical composition for detection of arteriosclerotic plaque.

This invention relates to the use of binding molecules for the extra-domain B (ED-B) of fibronectin, for example of labeled antibodies or antibody fragments against the ED-B domain, such as, for example, L19 derivatives, as diagnostic reagents for the detection of arteriosclerotic processes, in particular of arteriosclerotic plaque.

Arteriosclerosis is a change in blood vessels, which develops over many years and first proceeds undetected. The development of arteriosclerosis in this case proceeds over various stages. If it results in an injury to the endothelial cell layer of the arterial wall or non-adhering surface thereof because of mechanical or chemical trauma, the change in the normal blood flow resulting therefrom promotes the adherence and aggregation of blood platelets, in particular on the rami and branches of the arterial reticulum, which can result in the formation of blood clots, so-called thrombi, in the arterial walls. Over time, the accumulation of fatty layers results in a collection of foam cells, which are formed because of thrombi formation from monocytes of the cellular defense system, in a continuous cell invasion, cholesterol deposit, expansion of the smooth muscles as well as formation of additional binding tissue, by which it results in increasingly greater injuries. These advanced lesions ultimately represent the so-called arteriosclerotic plaque, which is found on the inside vascular wall, where they swell and concentrate by evaporation in the interior space of the artery. Later on, the arteriosclerotic plaques are then quickly coated with a thick layer of binding tissue. Over time, these plaques calcify, and this results in further changes, such as, e.g., tears or bleeding, which can result in a partial or total occlusion of the artery. As soon as no more blood can flow, the tissues and cells that are located behind are excluded from the supply. As a result of the stenosis, myocardial infarctions as well as attacks of angina pectoris, but also strokes, macular degeneration in the eye or thromboses can occur.

Arteriosclerosis develops quietly and stealthily and does not produce any symptoms for a long time. Only once the vascular diameter is increasingly reduced by the progressive formation of the arteriosclerotic plaque do the symptoms slowly, but steadily develop. Since calcifications of the arteriosclerotic plaque that have already occurred cannot be degraded and elasticity cannot be returned to the rigid arterial walls resulting therefrom, but the progress of the disease can be considerably slowed with information thereof, arteriosclerosis detection processes that can be performed easily with high sensitivity and specificity are of special importance.

Up until now, various methods of study were developed for diagnosis of arteriosclerosis, among them contrast-medium-enhanced angiography, multi-layer spiral CT, Doppler sonography, magnetic resonance tomography and electron ray tomography.

The contrast-medium-enhanced angiography is a method of study to visualize blood vessels by radiology. Depending on which organ or which body region is to be visualized, a hypodermic needle or a catheter is inserted into an artery, vein or into the tissue in local anesthesia. Then, a contrast medium or marker is injected, and the corresponding body region is x-rayed. In this case, both arteries, veins and lymph drainage pathways are described, by which indications on the type and the extent of the disease are possible. This method undergoes a significant limitation, however, by the available contrast media and markers. The latter make possible only a relatively unspecific visualization of the space in which they are found, such as, e.g., the blood space, by which the detection of the arteriosclerotic plaque is significantly hampered.

The multi-layer spiral CT represents an alternative to the contrast-medium-enhanced angiography. In addition to the valid documentation of calcified arteriosclerotic plaques, it offers in addition the advantage of high-resolution contrast-medium-enhanced CT angiography and thus also makes possible the visualization of uncalcified plaque. Initial clinical studies, however, show already clear limitations of the method, such that an unreflected clinical use in general cannot be recommended.

Doppler sonography is an ultrasound study in which the Doppler process is used and employs the diagnosis of heart diseases. By Doppler sonography, data on direction and speed of the blood flow are obtained, by which constructions of the hollow spaces of arteries can be detected. A visualization of arteriosclerotic plaque tissue is not possible, however, with Doppler sonography.

By using various pulse sequences, magnetic resonance tomography makes possible a visualization of arteriosclerotic plaque tissue as well as a tissue characterization of the individual plaque components. The use of this method for primary prevention requires, however, still considerable further development.

With the electron ray tomography, a process is available that allows a non-invasive determination of the extent of the coronary arteriosclerosis. By means of electron ray tomography, the progression of the coronary arteriosclerosis can thus also be considered. A drawback of the method is, however, that it is very expensive and requires the purchase of an electron ray tomograph.

The object of this invention is consequently to provide an alternative process for detection of arteriosclerotic plaque in arterial walls that has a high sensitivity and specificity and makes possible a simple, quick and economical primary prevention.

This object is achieved according to the invention by use of binding molecules against the ED-B of fibronectin for detection of arteriosclerotic processes, in particular for detection of arteriosclerotic plaque, including uncalcified and/or calcified plaque.

Binding molecules for the ED-B domains of fibronectin, a sequence of 91 amino acids, which is inserted by alternative splicing into the fibronectin molecule (Castellani et al. (1994), Int. J. Cancer 59, 612-618), are already described in WO 97/45544, WO 01/62800 and WO 03/055917. Preferred binding molecules are molecules that bind directly and specifically to the ED-B domains, such as, for example, antibodies against the ED-B domains or fragments of such antibodies, for example antibody fragments that can be obtained by proteolytic cleavage, e.g., Fab-, Fab′-, F(ab)₂ fragments, etc., or recombinant antibody fragments, e.g., single-chain Fv fragments. The ED-B-binding molecules are preferably used as conjugates with labeling groups that are suitable for diagnostic applications.

A preferred embodiment of the invention relates to the use of antibody L19 or fragments of this antibody (L19 derivatives), which are present as conjugates with labeling groups, for the production of a pharmaceutical composition for detection of arteriosclerotic plaque. Surprisingly enough, with the aid of studies that are based on this invention, it was possible to determine that the arteriosclerotic plaque can be diagnosed in a highly specific manner and with high sensitivity by binding labeled L19 derivatives.

L19 is the scFv fragment (scFv: single chain antibody fragment) of a monoclonal antibody against the extra-domain B (ED-B) of fibronectin and has the following amino acid sequence (SEQ ID NO.1):

(VH):
EVQLLESGGG LVQPGGSLRL SCAASGFTFS
SFSMSWVRQA PGKGLEWVSS ISGSSGTTYY
ADSVKGRFTI SRDNSKNTLY LQMNSLRAED
TAVYYCAKPF PYFDYWGQGT LVTVSS
(Linker):
GDGSSGGSGG ASTG
(VL):
EIVLTQSPGT LSLSPGERAT LSCRASQSVS
SSFLAWYQQK PGQAPRLLIY YASSRATGIP
DRFSGSGSGT DFTLTISRLE PEDFAVYYCQ
QTGRJPPTFG QGTKVEIK

L19 is already mentioned on various occasions in the prior art. Tarli et al. (Blood, Vol. 94, No. 1 (1999), pp. 192-198) thus describe the biodistribution of the highly affine human ¹²⁵I-labeled L19 in tumor-bearing mice with advanced angiogenesis in the area of the tumor tissue. In addition, WO 01/62800 discloses the use of radiolabeled conjugates, which comprise the scFv-fragment L19, for detecting and for treating angiogenesis. The use of labeled L19 derivatives for detection of arteriosclerotic plaque is neither disclosed nor suggested in the prior art, however.

The subject of this invention therefore relates in particular to the use of a labeled L19 derivative, comprising
(aa) at least one antigen binding site for the extra-domain B (ED-B) of fibronectin comprising the complementarity-determining regions HCDR3 and/or LCDR3, shown in Table 1, or a variant thereof, which exhibits a deletion, insertion and/or substitution of up to 5 amino acids in the HCDR3 region and up to 6 amino acids in the LCDR3 region, whereby the antigen binding site exhibits the same function as the native L19 shown in SEQ ID NO. 1,
(ab) at least one antigen binding site for the extra-domain B (ED-B) of fibronectin comprising the complementarity-determining regions HCDR1, HCDR2, HCDR3, LCDR1, LCDR2 and LCDR3, shown in Table 1, or a variant thereof, which exhibits a deletion, insertion and/or substitution of up to 3 amino acids in the HCDR1 region, of up to 8 amino acids in the HCDR2 region, of up to 5 amino acids in the HCDR3 region, of up to 6 amino acids in the LCDR1 region, of up to 4 amino acids in the LCDR2 region and of up to 6 amino acids in the LCDR3 region, whereby the antigen binding site exhibits the same function as the native L19 shown in SEQ ID NO.1, or
(ac) at least one antigen binding site for the extra-domain B (ED-B) of fibronectin comprising the sequence of the native L19, shown in SEQ ID NO. 1, or a variation thereof, which exhibits a deletion, insertion and/or substitution of up to 30 amino acids, whereby the antigen binding site exhibits the same function as the native L19 shown in SEQ ID NO. 1, and optionally
(ba) an amino acid sequence Xaa₁-Xaa₂-Xaa₃-Cys (SEQ ID NO. 2), whereby Xaa₁, Xaa₂, and Xaa₃, independently of one another, represent any naturally occurring amino acid,
(bb) an amino acid sequence Xaa₁-Xaa₂-Xaa₃-Cys-Xaa₄ (SEQ ID NO. 3), whereby Xaa₁, Xaa₂, Xaa₃, and Xaa₄, independently of one another, represent any naturally occurring amino acid,
(bc) an amino acid sequence (His)ₙ (SEQ ID NO. 4), whereby n is an integer from 4 to 6, or
(bd) an amino acid sequence that comprises the sequence shown in SEQ ID NO. 5, SEQ ID NO. 6 or SEQ ID NO. 7,
whereby the C-terminus of (aa), (ab), or (ac) is optionally bonded via a peptide bond to the N-terminus of (ba), (bb), (bc) or (bd),
   for the production of a pharmaceutical composition for detecting arteriosclerotic plaque.

Within the scope of this invention, the labeled L19 derivative comprises an N-terminal antigen binding site for the extra-domain B (ED-B) of fibronectin selected from the antigen binding sites (aa), (ab) or (ac) and optionally a C-terminal amino acid sequence selected from the amino acid sequences (ba), (bb), (bc) or (bd), whereby the antigen binding site exhibits the same function as the native L19 shown in SEQ ID NO. 1. According to this invention, this means that the antigen binding sites (aa), (ab) and (ac) of the labeled L19 derivative have a binding constant to arteriosclerotic plaque that is essentially identical to the native scFv-fragment L19 shown in SEQ ID NO. 1. In particular, the antigen binding sites (aa), (ab) and (ac) mediate a bond between the labeled L19 derivative and the arteriosclerotic plaque, whereby the complex that consists of labeled L19 derivative and arteriosclerotic plaque exhibits a dissociation constant in the subnanomolar range (e.g., less than 10⁻⁹ M). The dissociation constant of the complex that consists of labeled L19 derivative and arteriosclerotic plaque preferably lies in the same range as the dissociation constant of the complex that consists of the L19 derivative and the antigen ED-B fibronectin, described in WO 99/58570.

According to this invention, the antigen binding sites for the extra-domain B (ED-B) of fibronectin of the labeled L19 derivative (aa) or (ab) comprise the complementarity-determining regions HCDR3 and/or LCDR3 or HCDR1, HCDR2, HCDR3, LCDR1, LCDR2 and LCDR3, shown in Table 1. Within the scope of this invention, the complementarity-determining regions HCDR1, HCDR2, HCDR3, LCDR1, LCDR2 and LCDR3 are defined as follows:

TABLE 1

| | | | |
|---|---|---|---|
| | | | Maximum |
| | CDR Length⁽²⁾ | | (Preferred) |
| Region⁽¹⁾ | (in Amino Acids) | Sequence | Variations |

| | | | |
|---|---|---|---|
| HCDR1 | 5 | S F S M S | 3 (2,1) |
| | | (SEQ ID NO. 8) | |
| HCDR2 | 17 | S I S G S S G T T Y Y A D S V K G | 8 (7,6,5,4,3,2,1) |
| | | (SEQ ID NO. 9) | |
| HCDR3 | 7 | P F P Y F D Y | 5 (4,3,2,1) |
| | | (SEQ ID NO. 10) | |
| LCDR1 | 12 | R A S Q S V S S S F L A | 6 (5,4,3,2,1) |
| | | (SEQ ID NO. 11) | |
| LCDR2 | 7 | Y A S S R A | 4 (3,2,1) |
| | | (SEQ ID NO. 12) | |
| LCDR3 | 10 | C Q Q T G R I P P T | 6 (5,4,3,2,1) |
| | | (SEQ ID NO. 13) | |

⁽¹⁾HCDRx: Complementarity-determining region x the heavy antibody chain; LCDRx: complementarity-determining region x the light antibody chain.
⁽²⁾CDR length: Length of the complementarity-determining region.

In addition to the complementarity-determining regions defined in Table 1, the antigen binding sites for the extra-domain B (ED-B) of fibronectin of the labeled L19 derivative (aa) or (ab) can also comprise variants of these regions. According to the invention, a variant of the HCDR1 region comprises a deletion, insertion and/or substitution of up to 3 amino acids in the HCDR1 region, i.e., a deletion, insertion and/or substitution of 1, 2 or 3 amino acids relative to the sequence (SEQ ID NO. 8) shown in Table 1. A variant of the HCDR2 region comprises a deletion, insertion and/or substitution of up to 8 amino acids in the HCDR2 region, i.e., a deletion, insertion and/or substitution of 1, 2, 3, 4, 5, 6, 7 or 8 amino acids relative to the sequence (SEQ ID NO. 9) shown in Table 1. Moreover, a variant of the HDCR3 region comprises a deletion, insertion and/or substitution of up to 5 amino acids in the HCDR3 region, i.e., a deletion, insertion and/or substitution of 1, 2, 3, 4 or 5 amino acids relative to the sequence (SEQ ID NO. 10) shown in Table 1. A variant of the LCDR1 region, however, comprises a deletion, insertion and/or substitution of up to 6 amino acids in the LCDR1 region, i.e., a deletion, insertion and/or substitution of 1, 2, 3, 4, 5 or 6 amino acids relative to the sequence (SEQ ID NO. 11) shown in Table 1. In addition, a variant of the LCDR2 region comprises a deletion, insertion and/or substitution of up to 4 amino acids in the LCDR2 region, i.e., a deletion, insertion and/or substitution of 1, 2, 3 or 4 amino acids relative to the sequence (SEQ ID NO. 12) shown in Table 1. A variant of the LCDR3 region comprises a deletion, insertion and/or substitution of up to 6 amino acids in the LCDR3 region, i.e., a deletion, insertion and/or substitution of 1, 2, 3, 4, 5 or 6 amino acids relative to the sequence (SEQ ID NO. 13) shown in Table 1.

According to this invention, the antigen binding site for the extra-domain B (ED-B) of fibronectin of the labeled L19 derivative (ac) comprises the sequence of native L19, shown in SEQ ID NO. 1, or a variation thereof, which exhibits a deletion, insertion and/or substitution of up to 30 amino acids, i.e., a deletion, insertion and/or substitution of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids relative to the sequence shown in SEQ ID NO. 1.

The amino acid sequences (ba), (bb) or (bc) of the labeled L19 derivative comprise the sequences Xaa₁-Xaa₂-Xaa₃-Cys (SEQ ID NO. 2), Xaa₁-Xaa₂-Xaa₃-Cys-Xaa₄ (SEQ ID NO. 3) or (HIS)ₙ (SEQ ID NO. 4).

In a preferred embodiment of this invention, the amino acid sequence (ba) Xaa₁-Xaa₂-Xaa₃-Cys (SEQ ID NO. 2) is the sequence Gly-Gly-Gly-Cys (SEQ ID NO. 14) or Gly-Cys-Gly-Cys (SEQ ID NO. 15). Especially preferred is the sequence Gly-Gly-Gly-Cys (SEQ ID NO. 14).

In another preferred embodiment of this invention, the amino acid sequence (bb) Xaa₁-Xaa₂-Xaa₃-Cys-Xaa₄ (SEQ ID NO. 3) is the sequence Gly-Gly-Gly-Cys-Ala (SEQ ID NO. 16) or Gly-Cys-Gly-Cys-Ala (SEQ ID NO. 17). Especially preferred is the sequence Gly-Gly-Gly-Cys-Ala (SEQ ID NO. 16).

In another preferred embodiment of this invention, the amino acid sequence (bc) (His)ₙ (SEQ ID NO. 4) is the sequence (His)₆ with n equal to 6 (SEQ ID NO. 18).

In another preferred embodiment of this invention, the N-terminus of (aa), (ab) or (ac) is optionally connected via a peptide bond to the C-terminus of a linker amino acid sequence. The linker amino acid sequence preferably has a length of up to 30 amino acids, preferably up to 25 amino acids, and especially preferably up to 22 amino acids. Especially preferred is the linker amino acid sequence, which is the sequence shown in SEQ ID NO. 19.

According to this invention, especially preferred labeled L19 derivatives comprise the sequences shown in SEQ ID NO. 1 (native L19), SEQ ID NO. 20 (AP38), SEQ ID NO. 21 (AP39), SEQ ID NO. 22 (L19-GlyCysGlyCys), SEQ ID NO. 23 (L19-GlyCysGlyCysAla), SEQ ID NO. 24 (ZK225293), SEQ ID NO. 25 (ZK217691/217695), SEQ ID NO. 26 (ZK210917) and SEQ ID NO. 27 (ZK248219/248220).

The binding molecule for the ED-B domain preferably is present in the form of a conjugate with a labeling substance. As labeling substances, all labeling substances that are suitable for diagnostic applications, especially diagnostic applications in vivo, are suitable, for example radiolabeling substances, or for non-radioactive detecting methods, e.g., labeling substances that are suitable for magnetic resonance processes.

Processes for introducing labeling substances in polypeptides, peptides and especially scFv fragments are well known in the prior art. The binding molecule is preferably labeled with a radioisotope, e.g., a radioisotope of iodine (I), indium (In), technetium (Tc) and rhenium (Re). Especially preferred are the radioisotopes ¹²⁵I, ¹¹¹In, ¹⁸⁶Re, ¹⁸⁸Re, ^{94m}Tc or ^{99m}Tc.

In a preferred embodiment of this invention, an antibody fragment, e.g., an L19 derivative in reduced form, is used. Within the scope of this invention, the term “reduced form” means that the fragment is present in monomeric form and not, for example, in dimeric or multimeric form that is mediated by intermolecular disulfide bridges. The reduced form of the antibody fragment is preferably obtained by adding a suitable reducing agent. Suitable reducing agents are well known in the prior art and comprise TCEP (tris(2-carboxyethyl)phosphine) and 1,4-dimercapto-2,3-butanediols.

In addition, this invention provides that the pharmaceutical composition, in addition to the binding molecule, optionally contains physiologically compatible adjuvants, vehicles and/or diluents. Suitable adjuvants, vehicles and/or diluents are best known to one skilled in the art in the field of pharmaceutical chemistry.

The detection of arteriosclerotic plaque is preferably carried out within the scope of this invention by injecting the pharmaceutical composition, which comprises the ED-B-binding molecule, into a vein and/or artery of a patient to be examined and detecting the labeled ED-B-binding molecule that is bonded to the arteriosclerotic plaque—if present. If a radioisotope-labeled binding molecule is used, the detection can be carried out by scintigraphy. Myocardial infarctions as well as attacks of angina pectoris, but also strokes, macular degeneration in the eye and/or thromboses can be prevented by the early detection of arteriosclerotic plaque according to this invention.

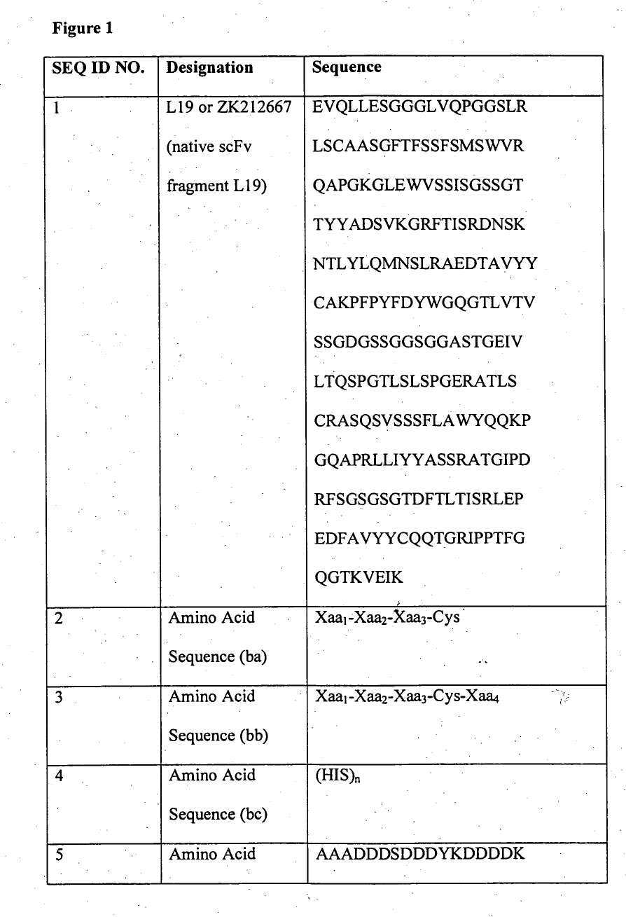

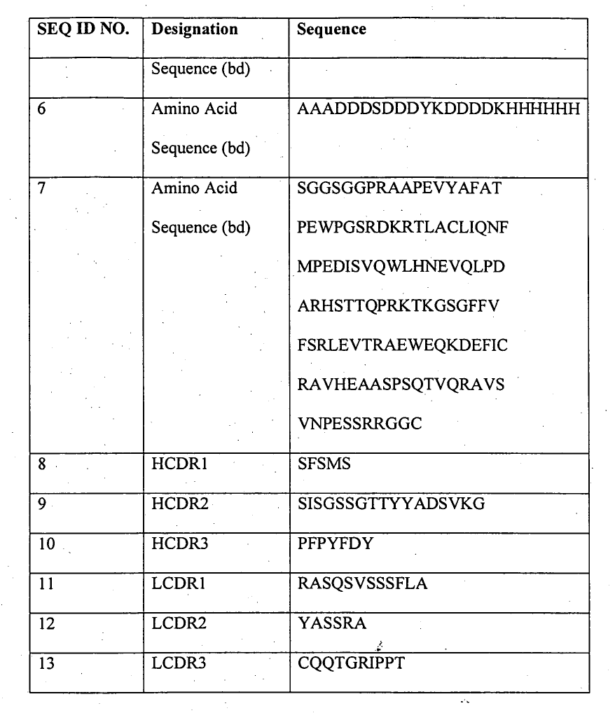

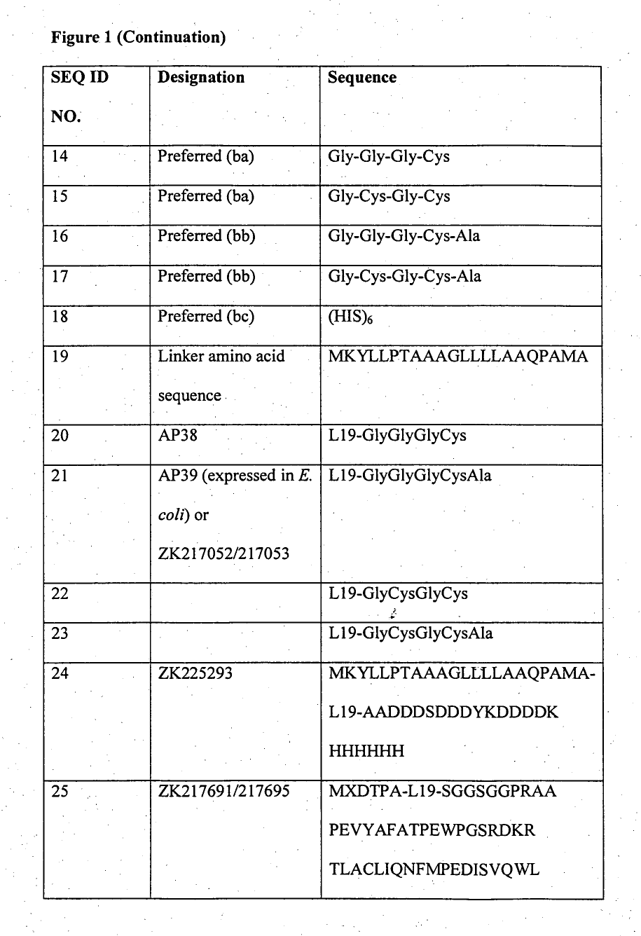

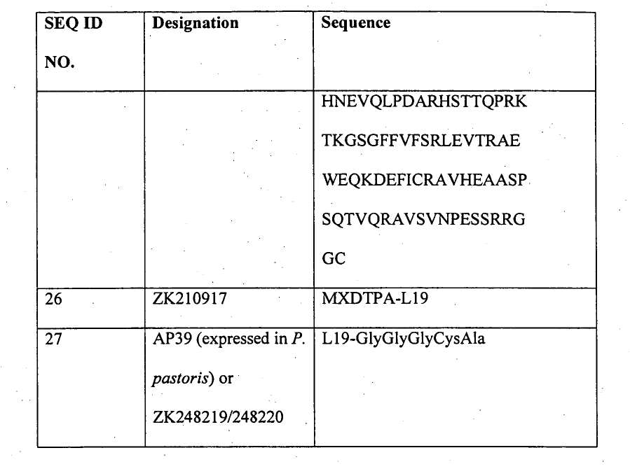

In addition, this invention is explained in more detail by FIG. 1 and the examples below.

### EXAMPLE

### Example 1

### Production of L19 Derivatives

The production of L19 derivatives is carried out as described in WO 03/055917, to whose content reference is made herein.

### Example 2

### Labeling of L19 Derivatives with the Aid of Radioisotopes

The production of labeled L19 derivatives is carried out as described in WO 03/055917, to whose content reference is made herein.

### Example 3

### Study of the Binding of Various, Labeled L19 Derivatives to Arteriosclerotic Vascular Specimens of WHHL Rabbits in a Special in Vitro Perfusion Apparatus

To study the suitability of various, labeled L19 derivatives, an in vitro perfusion apparatus (Ussing chamber) was used. This perfusion apparatus contained vascular specimens from the aorta of WHHL rabbits (Watanabe heritable hyperlipidemic rabbits).

Owing to a genetic defect in certain sections of the aorta, these WHHL rabbits develop arteriosclerotic plaque. Vascular specimens from these arteriosclerotic sections of the aorta were therefore used as models for the disease arteriosclerosis in humans. As a comparison control, vascular specimens of non-arteriosclerotic sections of the aorta in each case from the same rabbit were used.

The vascular specimens were positioned in the vascular apparatus in such a way that the respectively labeled L19 derivative to be studied could bind only to the luminal side of the aorta. A solution of the labeled L19 derivative was in this case perfused with the aid of a peristaltic pump at a rate of 1 ml/min. The perfusion was performed over 20 minutes at room temperature. The volume of the perfusion circuit was 9 ml. In this volume, the labeled L19 derivative according to the invention was contained in the amount indicated in Table 2.

After perfusion is terminated, the amount of the labeled L19 derivative to be studied, bonded to the arteriosclerotic plaque of the aorta, was determined with the aid of a γ-counter (Elscint SP4 HR γ-camera). Based on the ratio of the amount of bonded labeled L19 derivative to the arteriosclerotic and non-arteriosclerotic sections of the aorta, the rating factor of the respective labeled L19 derivative is determined.

TABLE 2

| L19 Derivative | |
|---|---|
| to be Examined: | Amount Used and Labeling: |
| ZK225293 | 0.3375 pmol labeled with 0.61 MBq of ¹²⁵I |
| ZK212667 | 0.482 pmol labeled with 0.945 MBq of ¹²⁵I |
| ZK2176691/217695 | 584.775 pmol labeled with 1.61 MBq of ¹¹¹In |
| ZK210917 | 283.68 pmol labeled with 1.5 MBq of ¹¹¹In |
| ZK217052/217053 | 72.648 pmol labeled with 3.0 MBq of ^{99m}Tc |

### Example 3.1

### Study of the ¹²⁵I-Labeled L19 Derivative ZK225293

The study of the suitability of ZK225293 (SEQ ID NO. 24) was performed as described in Example 3. The rating factor for ZK225293 determined in the study was 4.5. The result of this study shows the excellent potential of the labeled L19 derivative for testing arteriosclerotic plaque and thus for diagnosis of arteriosclerosis in arteries.

### Example 3.2

### Study of the ¹²⁵I-Labeled L19 Derivative ZK212667

The study of the suitability of ZK212667 (L19; SEQ ID NO. 1) was performed as described in Example 3. The rating factor for ZK212667, determined in the study, was 2.8. The result of this study shows the excellent potential of the labeled L19 derivative for detecting arteriosclerotic plaque and thus for diagnosis of arterioscleroses in arteries.

### Example 3.3

### Study of the ¹¹¹In-Labeled L19 Derivative ZK21769/217695

The study of the suitability of ZK217691/217695 (SEQ ID NO. 25) was performed as described in Example 3. The rating factor for ZK2176691/217695, determined in the study, was 8.7. The result of this study shows the excellent potential of the labeled L19 derivative for detecting arteriosclerotic plaque and thus for the diagnosis of arterioscleroses in arteries.

### Example 3.4

### Study of the ¹¹¹In-Labeled L19 Derivative ZK210917

The study of the suitability of ZK210917 (SEQ ID NO. 26) was performed as described in Example 3. The rating factor for ZK210917, determined in the study, was 3.4. The result of this study shows the excellent potential of the labeled L19 derivative for detecting arteriosclerotic plaque and thus for the diagnosis of arteriosclerosis in arteries.

### Example 3.5

### Study of the ^{99m}Tc-Labeled L19 Derivative ZK217052/217053

The study of the suitability of ZK217052/217053 (SEQ ID NO. 21) was performed as described in Example 3. The rating factor for ZK217052/217053, determined in the study, was 4.8. The result of this study shows the excellent potential of the labeled L19 derivative for detecting arteriosclerotic plaque and thus for the diagnosis of arterioscleroses in arteries.

### Example 4

### Study of the Imaging of Arteriosclerotic Plaque with the Aid of ^{99m}Tc-Labeled L19 Derivative ZK248219/248220 in WHHL Rabbits in Vivo

The study of the suitability of ZK248219/248220 (SEQ ID NO. 27) was performed in vivo on a WHHL rabbit. Owing to a genetic defect, these WHHL rabbits developed arteriosclerotic plaque in certain sections of the aorta and were therefore used as models for the disease arteriosclerosis in humans.

Under anesthesia (Rompun/Ketavet (1:2), 1 ml/kg of body weight i.m.), 41 MBq of the ^{99m}Tc -labeled L19 derivative ZK248219/248220) was administered to the test animal (3.4 kg of body weight) in the marginal vein of the ear. Over a period of 5 hours, whole-body scintigrams were recorded with the γ-counter (Elscint SP4 HR γ-camera). After 5 hours, the test animal was sacrificed, and its aorta was studied by autoradiography to determine the exact distribution of the activity bonded to the aorta.

The result of the imaging study shows a clear visualization of the aortic arch up until the time post injection. The autoradiographic study confirms that in the aortic arch of the test animal, an activity concentration that is higher by a factor of 12 than in the plaque-free, abdominal aortic areas is present. The result of this study thus shows quite clearly the excellent potential of the labeled L19 derivative ZK248219/248220 for the diagnosis of arteriosclerotic plaque.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited herein and of corresponding German application No. 10348319.5, filed Oct. 17, 2003 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of binding molecules against the extra-domain B of fibronectin for the production of a pharmaceutical composition for detection of arteriosclerotic plaque.

2. Use according to claim 1, characterized in that the binding molecules are selected from antibodies and fragments thereof.

3. Use according to claim 1, wherein the binding molecules carry a labeling group.

4. Use according to claim 1, wherein the binding molecules are selected from 19-derivatives, comprising
(aa) at least one antigen binding site for the extra-domain B (ED-B) of fibronectin comprising the complementarity-determining regions HCDR3 and/or LCDR3, shown in Table 1, or a variant thereof, which exhibits a deletion, insertion and/or substitution of up to 5 amino acids in the HCDR3 region and of up to 6 amino acids in the LCDR3 region, whereby the antigen binding site exhibits the same function as the native L19 shown in SEQ ID NO. 1,
(ab) at least one antigen binding site for the extra-domain B (ED-B) of fibronectin comprising the complementarity-determining regions HCDR1, HCDR2, HCDR3, LCDR1, LCDR2 and LCDR3, shown in Table 1, or a variant thereof, which exhibits a deletion, insertion and/or substitution of up to 3 amino acids in the HCDR1 region, of up to 8 amino acids in the HCDR2 region, of up to 5 amino acids in the HCDR3 region, of up to 6 amino acids in the LCDR1 region, of up to 4 amino acids in the LCDR2 region and of up to 6 amino acids in the LCDR3 region, whereby the antigen binding site exhibits the same function as the native L19, shown in SEQ ID NO. 1, or
(ac) at least one antigen binding site for the extra-domain B (ED-B) of fibronectin comprising the sequence of the native L19, shown in SEQ ID NO. 1, or a variation thereof, which exhibits a deletion, insertion and/or substitution of up to 30 amino acids, whereby the antigen binding site exhibits the same function as the native L19 shown in SEQ ID NO. 1,
and optionally
(ba) an amino acid sequence Xaa₁-Xaa₂-Xaa₃-Cys (SEQ ID NO. 2), whereby Xaa₁, Xaa₂ and Xaa₃, independently of one another, represent any naturally occurring amino acid,
(bb) an amino acid sequence Xaa₁-Xaa₂-Xaa₃-Cys-Xaa4 (SEQ ID NO. 3), whereby Xaa₁, Xaa₂, Xaa₃, and Xaa₄, independently of one another, represent any naturally occurring amino acid,
(bc) an amino acid sequence (His)ₙ (SEQ ID NO. 4), whereby n is an integer from 4 to 6, or
(bd) an amino acid sequence that comprises the sequence shown in SEQ ID NO. 5, SEQ ID NO. 6 or SEQ ID NO. 7,
whereby the C-terminus of (aa), (ab) or (ac) optionally is bonded via a peptide bond to the N-terminus of (ba), (bb), (bc) or (bd).

5. Use according to claim 4, wherein the amino acid sequence Xaa₁-Xaa₂-Xaa₃-Cys is the sequence Gly-Gly-Gly-Cys (SEQ ID NO. 14) or Gly-Cys-Gly-Cys (SEQ ID NO. 15).

6. Use according to claim 4, wherein the amino acid sequence Xaa₁-Xaa₂-Xaa₃-Cys-Xaa4 is the sequence Gly-Gly-Gly-Cys-Ala (SEQ ID NO. 16) or Gly-Cys-Gly-Cys-Ala (SEQ ID NO. 17).

7. Use according to claim 4, wherein n in the amino acid sequence (HIS)ₙ is 6 (SEQ ID NO. 18).

8. Use according to claim 4, wherein the N-terminus of (aa), (ab) or (ac) optionally is connected via a peptide bond to the C-terminus of a linker amino acid sequence.

9. Use according to claim 8, wherein the linker amino acid sequence exhibits a length of up to 30 amino acids.

10. Use according to claim 8, wherein the linker amino acid sequence is the sequence shown in SEQ ID NO. 19.

11. Use according to one of claim 4, wherein the labeled L19 derivative comprises the sequence shown in SEQ ID NO. 1, SEQ ID NO. 20, SEQ ID NO. 21, SEQ ID NO. 22, SEQ ID NO. 23, SEQ ID NO. 24, SEQ ID NO. 25, SEQ ID NO. 26 or SEQ ID NO. 27.

12. Use according to claim 1, wherein the binding molecule is labeled with a radioisotope.

13. Use according to claim 12, wherein the radioisotope is selected from radioisotopes of iodine (I), indium (In), technetium (Tc), and rhenium (Re).

14. Use according to claim 12, wherein the radioisotope is ¹²⁵I, ¹¹¹In, ¹⁸⁶Re, ¹⁸⁸Re, ^{94m}Tc, or ^{99m}Tc.

15. Use according to claim 1, wherein the binding molecules are selected from antibody fragments, in particular L19 derivatives in reduced form.

16. Use according to claim 1, wherein the pharmaceutical composition contains additional physiologically compatible adjuvants, vehicles and/or diluents.

17. Use according to claim 1, wherein the composition is provided for injection in a vein and/or artery of a patient.

18. Use according to claim 1, wherein the composition contains a radiolabeled binding molecule that is suitable for detection.

19. Use according to claim 1 for prevention of myocardial infarctions as well as attacks of angina pectoris, but also strokes, macular degeneration in the eye or thromboses.

20. Process for the detection of arteriosclerotic plaque, comprising the administration of a binding molecule against the extra-domain B of fibronectin in a diagnostically adequate amount to a patient who is to be examined, in particular a human patient, and determination of the location of the binding molecule in the blood vessels of the patient.
